(19) United States
(12) Patent Application Publication
(10) Pub. No.: US 2023/0000443 A1
(43) Pub. Date: Jan. 5, 2023

(54) LAYER STRUCTURE OF A SENSOR FOR CAPACITIVE MEASUREMENT OF BIOELECTRICAL SIGNALS

(71) Applicant: Siemens Healthcare GmbH, Erlangen (DE)

(72) Inventor: Ulrich BATZER, Spardorf (DE)

(73) Assignee: Siemens Healthcare GmbH, Erlangen (DE)

(21) Appl. No.: 17/851,351

(22) Filed: Jun. 28, 2022

(30) Foreign Application Priority Data
Jun. 30, 2021 (DE) 10 2021 206 856.1

(51) Int. Cl. A61B 5/00; A61B 5/318; A61B 5/369; G01R 31/58

(52) U.S. Cl. CPC A61B 5/7221; A61B 5/318; A61B 5/369; A61B 5/7225; G01R 31/58; A61B 2560/0276; A61B 2562/222

(57) ABSTRACT

A signal measurement circuit comprises: a sensor electrode layer connected via a sensor cable to a measurement amplifier circuit; an active shielding layer, which runs along a side of the sensor electrode layer that faces away from the patient; and a first insulating layer that runs between the sensor electrode layer and the active shielding layer. The sensor electrode layer and the active shielding layer are embodied to be electrically conductive.

### CROSS-REFERENCE TO RELATED APPLICATION(S)

The present application claims priority under 35 U.S.C. § 119 to German Patent Application No. DE 10 2021 206 856.1, filed Jun. 30, 2021, the entire contents of which are incorporated herein by reference.

### FIELD

Embodiments of the present invention relate to a signal measurement circuit in a layer structure for a differential voltage measurement system for measuring bioelectrical signals from a patient. In particular this involves electrically conductive electrode or shielding layers being insulated from one another via insulating layers.

### BACKGROUND

Voltage measurement systems, in particular differential voltage measurement systems, for measuring bioelectrical signals are used in medicine, for example, for measuring electrocardiograms (ECGs), electroencephalograms (EEGs) or electromyograms (EMGs).

The measurement of cardiac activity with the aforementioned voltage measurement systems is necessary in particular for imaging the heart, in order to adjust the imaging procedure to the very pronounced movement of the heart during the heartbeat. For this purpose, the convention is to use sensors that have to be attached to the patient's body. One option for heartbeat measurement is a capacitive ECG, in which an ECG signal is picked up capacitively only, without having direct contact between the patient and the sensor, in particular through the patient's clothing. In order to achieve a good signal quality of the heartbeat signal, the amplitude of the measured signal must preferably be high. This can be achieved by a high capacitance between the patient and the sensor. The capacitance can be influenced directly by the size of the coupling area between the sensor and the patient. The greater the coupling area, the greater is also the capacitance achieved.

### SUMMARY

The inventors have discovered that a challenge exists in embodying the capacitive sensors such that they are suitable for use in a clinical environment. Yet, the capacitive sensors have to meet demanding specifications with respect to cleanability, disinfectability and mechanical robustness. Furthermore, the capacitive sensors must not impair medical image data acquisition, for the triggering of which the bioelectrical signal that has been acquired can be used, that is, the sensors further must in particular be x-ray transparent or MR-invisible. In addition, in the context of an adequate measured signal quality, the capacitive sensors for a desired positioning on the patient must be embodied to be flexible or moldable and must have good triboelectric properties.

Capacitive ECG arrangements that are incorporated in layers in conductive textiles are known, with conductivity being achieved, for example, via a vapor deposition process with conductive particles. However, the use of textiles as an integral component of a sensor element complicates the cleaning process. Furthermore, textiles are not x-ray-transparent and therefore are not suitable for triggering a random medical data acquisition. In addition, ECG arrangements with metallic surfaces are known, but these also lack transparency to x-rays.

In contrast, embodiments of the present invention meet the requirements made by a clinical environment at least with respect to watertightness, cleanability, robustness and/or imaging properties.

At least this object is achieved by a signal measurement circuit according to one or more embodiments of the present invention and by a differential voltage measurement system. Further particularly advantageous variants and further developments of embodiments of the present invention will emerge from the description that follows, with it also being possible for individual features of various exemplary embodiments or variants to be combined to form new exemplary embodiments or variants.

A first aspect of embodiments of the present invention relates to a signal measurement circuit in a layer structure for a differential voltage measurement system for measuring bioelectrical signals from a patient. The signal measurement circuit comprises a sensor electrode layer that is connected via a sensor cable to a measurement amplifier circuit. The sensor electrode is therefore embodied according to embodiments of the present invention as a flat electrode or in layers. The signal measurement circuit further comprises an active shielding layer, which runs along the side of the sensor electrode layer that faces away from the patient. The active shielding is also embodied according to embodiments of the present invention to be flat or in layers. The sensor electrode layer and active shielding layer are each embodied to be electrically conductive. Between the sensor electrode layer and the active shielding layer runs a first insulating layer, likewise embodied to be flat, which prevents a short circuit from occurring between the sensor electrode layer and the active shielding layer.

The sensor cable is used to transmit the measured signals that have been acquired via the sensor electrode to the measurement amplifier circuit.

The measurement amplifier circuit preferably comprises an operational amplifier, which can be embodied as a “trailer”. This means that the negative input from the operational amplifier, also known as an inverting input, is coupled to the output from the operational amplifier, as a result of which a high virtual input impedance is generated on the positive input.

The active shielding layer covers the sensor electrode layer, and both layers preferably have the same basic area. In embodiments of the present invention, the active shielding layer is connected to the output of the operational amplifier. Its potential is controlled or adjustable. The potential of the active shielding layer is kept close to the potential of the sensor electrode layer, such that a flow of current from the sensor electrode layer onto the active shielding layer is prevented. The active shielding layer deflects any interference from the surrounding environment such that it cannot reach the sensor electrode layer and cannot couple into it. The active shielding layer is characterized by a high virtual input impedance.

The sensor electrode layer, the first insulating layer and the active shielding layer, and also the further layers optionally introduced below, together form a sensor element, which is positioned on top of or on the side of the patient for measured signal acquisition, with the sensor electrode layer orientated toward the patient.

Due to the layer-type design, in which the individual layers are considerably smaller in size in one spatial dimension than in the other two spatial dimensions, the signal measurement circuit is particularly thin and therefore can be easily molded onto a patient's body.

A second aspect of the present invention relates to a differential voltage measurement system for measuring bioelectrical measured signals from a patient. The voltage measurement system has at least two signal measurement circuits, each corresponding with a wanted signal pathway each comprising a sensor electrode. The differential voltage measurement system can also comprise more than two signal measurement circuits. At least one of the signal measurement circuits, preferably all the signal measurement circuits comprised in the system, is embodied as described above and hereafter. In particular, electrodes and/or shields in at least one signal measurement circuit are designed to be flat or in layers.

As already mentioned in the introduction, the differential voltage measurement system according to embodiments of the present invention acquires bioelectrical signals, for example from a human or animal patient. For this purpose, it has a number of measurement cables or wanted signal pathways. These connect, as individual cables for example, electrodes that are attached to the patient for the acquisition of the signals, with further components of the voltage measurement system, that is, in particular, an electronics unit that is used for the evaluation or display of the bioelectrical signals that have been acquired, in particular heartbeat signals.

Differential voltage measurement systems are known to a person skilled in the art with respect to their basic mode of functioning, which is why a more detailed explanation is dispensed with here. They can be embodied in particular as electrocardiograms (ECGs), electroencephalograms (EEGs) or electromyograms (EMGs).

In embodiments, the differential voltage measurement system can comprise a reference measurement circuit comprising a reference electrode. Reference electrodes or the associated reference measurement circuits are used to create a potential equalization between the patient and the ECG measurement device. In embodiments of the present invention, the reference measurement circuit likewise comprises a sensor cable and an operational amplifier. In embodiments of the present invention, the reference electrode is embodied as a separate, autonomous sensor element.

In embodiments, the differential voltage measurement system can comprise a ground electrode that is preferably embodied as a separate sensor element, via which the patient can be at least capacitively coupled with the earth potential during signal acquisition.

The differential voltage measurement system according to embodiments of the present invention therefore has at least one signal measurement circuit according to embodiments of the present invention. The differential voltage measurement system according to embodiments of the present invention accordingly shares the advantages of the signal measurement circuit according to embodiments of the present invention.

In advantageous embodiments, the signal measurement circuit further comprises a second electrically conductive shielding layer, which is arranged on the side of the active shielding layer that faces away from the sensor electrode and which is separated from this by an insulating layer. This passive, second shielding that covers the active shielding layer is used to intercept particularly strong electric fields, which would overload the operational amplifier mentioned in the introduction that controls the protective shield. In these embodiments, it is possible to shield effectively against even very strong electric interference fields. The second shielding layer is insulated from the active shielding layer by a second insulating layer. The additional layers provided here advantageously ensure an improvement in the measured signals acquired, without essentially having an influence on the overall height of the signal measurement circuit.

In other embodiments, the signal measurement circuit comprises an electrically conductive ground electrode layer, to connect the signal measurement circuit to the earth potential. The ground electrode is therefore also embodied here as a flat electrode or in layers and is arranged on the side of the second shielding layer that faces away from the sensor electrode layer. A third insulating layer separates the ground electrode layer from the second, passive shielding layer. A fourth insulating layer separates it from the surrounding environment.

The sensor electrode layer, the active shielding layer, the further shielding layer and/or the ground electrode layer are advantageously embodied to be equal in shape and size, as already mentioned in the introduction. The greater the sensor electrode area, the greater is the capacitance and hence the quality of the measured signal that has been acquired. The sizes of the basic areas of the aforementioned layers therefore advantageously range from 9 cm² to 64 cm². The basic shape of the layers can vary between round, rectangular, in particular square or suchlike. The aforementioned layers preferably have a size of 24 cm², then rectangular with the sides measuring 6 cm by 4 cm or 25 cm², then square with sides measuring 5 cm.

Particularly advantageous embodiments of the signal measurement circuit make provision for the first, second, third and/or fourth insulating layer to protrude over each of the layers to be insulated. Advantageously, all the insulating layers are embodied to be larger. This means that the basic shape of the first, second, third and/or fourth insulating layer is indeed the same as the basic shape of the sensor electrode layer, of the two shielding layers and/or of the ground electrode layer but has a greater basic area. The insulating layers therefore have a greater size than the electrode or shielding layers. For example, the insulating layers can protrude radially by 2 mm over the electrode and shielding layers where the basic shape is circular. This ensures that, even in the case of a flexible deformation or molding of the sensor element formed by the layer structure, short circuits between the electrically conductive electrode and shielding layers are avoided.

The signal measurement circuit according to embodiments of the present invention is embodied particularly advantageously when the layer thicknesses of the sensor electrode layer, of the active shielding layer, of the further passive shielding layer, of the ground electrode layer, of the first, second, third and/or fourth insulating layer have a layer thickness ranging from 50 μm to 500 μm. Particularly preferably, all the layers of the sensor element are embodied with a height in this range, for example 300 μm. Ideally, as a result, an overall height of the sensor element does not exceed a value of 4 mm, and in most cases is even thinner. The thinner the layer structure overall, the better the sensor element can be molded onto the patient's anatomy or the more flexible it is.

In advantageous embodiments, the sensor electrode layer, the active shielding layer, the further shielding layer and/or the first, second, third and/or fourth insulating layer is in each case fused or welded with one of the adjacent layers. This ensures a frictional connection between the layers and increases the stability of the sensor element formed by the layers; the insulation of the conductive electrode and shielding layers is additionally guaranteed. These advantages come into effect particularly when all the layers are each connected with their neighboring layers, as described.

The sensor electrode layer, the active shielding layer, the further shielding layer and/or the ground electrode layer is particularly advantageously embodied using a carbon particle-enriched plastic.

Here, the plastic base material ensures the mechanical stability in the event of at least partially elastic, in any case reversible, moldability of the electrode and shielding layers. The admixture of carbon advantageously ensures the electrical conductivity of these layers. The carbon particles can preferably be nanoparticles. Here, the filler content of the carbon admixture is dependent on the desired conductivity and on the type of carbon particles used. When selecting the carbon particles, it needs to be taken into account in particular that with a higher filler content the influencing of the mechanical material properties increases. In particular, an adequate conductivity can already be achieved, even where there is a very low filler content of a low volume percentage, by using carbon nanotubes. According to embodiments of the present invention, a maximum of 50% carbon is to be added.

In addition, in other embodiments, the base material for the electrode and shielding layers can be selected according to desired tribological properties. In particular, a material with tribological properties adjusted to cotton or cellulose can be used since these textiles are most frequently expected to be used in patient clothing and are therefore the supporting surface for the materials that form the sensor electrode layer.

Examples of the base material are in particular polyurethane (PU) or polyvinyl chloride (PVC).

Particularly advantageously, the sensor electrode layer is embodied such that it has a surface resistance ranging from 10 kΩ to 100 kΩ. This and all the following resistance data correspond with the requirements of DIN EN 61340-2-3 (VDE 0300-2-3), Electrostatics—Part 2-3: Test methods for determining the resistance and resistivity of solid materials used to avoid electrostatic charge accumulation (IEC 61340-2-3:2016). A desired surface resistance is preferably achieved by a corresponding filler content of carbon particles in the base material.

In further preferred embodiments of the signal measurement circuit, the first, second, third and/or fourth insulating layer likewise consists of a plastic. Preferably, all the insulating layers consist of a plastic. It is particularly advantageous in the context of production comprising many non-variant parts if all the layers of the sensor element are embodied using the same base material. In particular, it is possible in this way for the same tribological properties to also be achieved for all the layers comprised therein. In particular, this also results in a particularly homogeneous moldability. Of course, a different base material can be selected for individual or for all the insulating layers, for example, in particular the fourth external insulating layer, with care needing to be taken to ensure that the tribological properties are at least similar.

The base material for individual or for all the insulating layers should retain good or adequate insulating properties even with a humidity of 100% at up to 40° C. These ambient conditions occur when a sensor element is in electrical contact for a longer period through a patient. The base material of the insulating layers in preferred embodiments is therefore hydrophobic. This means that it is water-repellent and only minimally absorbs fluids, and as a result there is barely any change in its insulating properties.

Particularly preferably, the insulating layers are therefore formed from aliphatic or aromatic polyurethane (PU) or polyvinyl chloride (PVC).

Due to all the layers that form the sensor element being embodied from a flexibly moldable, thin, film- or slice-type material, a high degree of patient comfort can be achieved, since the sensor element can assume any desired shapes according to an individual patient's anatomy, is flexible and light and rarely wears out.

In embodiments of the signal measurement circuit, the first and/or the second insulating layer, that is, the insulating layers between the sensor electrode layer and the first passive shielding layer and between the two shielding layers, is embodied such that it has a volume resistance ranging from 50 MΩ to 50 GΩ. Both insulating layers are preferably embodied with such a volume resistance. The first insulating layer between the sensor electrode layer and the active shielding layer functions as a voltage divider for the measured bioelectrical signal.

In further embodiments of the signal measurement circuit, the third insulating layer is embodied such that it has a volume resistance ranging from 1 GΩ to 100 GΩ.

The third insulating layer between the passive shielding layer and the ground electrode layer positively suppresses the coupling of disruptive external electrical fields.

From the viewpoint of electrical safety, provision can be made in embodiments of the present invention for the third insulating layer, which insulates the ground electrode layer, to be able to resist a dielectric strength at 4 kV AC (alternating current) for example, for over 1 min.

In a particularly preferred embodiment of the signal measurement circuit, the sensor electrode layer has a segment that is surrounded on both sides by a first insulating layer and an active shielding layer and forms in this way a section of the sensor cable. In other words, the sensor electrode layer itself forms a section of the sensor cable. This section is embodied as a flat conductor element. For shielding and insulation purposes, the segment of the sensor electrode layer is covered on both sides with a first insulating layer and an active shielding layer. The section of the sensor cable that is formed in this way has very similar mechanical properties to the sensor element that is formed from the various layers and is therefore flexible, moldable and in particular flat. The section of the sensor cable that is formed in this way advantageously connects directly to the sensor element, that is, the segment of the sensor electrode layer and the remaining sensor electrode layer that pertains to the sensor element are embodied in one piece. The sensor cable section is therefore embodied with the same layers as the sensor element, wherein the materials for the individual layers are embodied according to the sensor element. In embodiments, the section of the sensor cable that is formed in this way can evolve into a further conventionally embodied form of cable.

In a further development of embodiments of the present invention, the sensor cable has a length ranging from 20 cm to 200 cm and a width ranging from 2 cm to 6 cm. In this way, the sensor cable section that is adjacent to the sensor element that is to be positioned on the patient is embodied in particular to be unobtrusive for or robust to interference signals and/or mechanical stress/load.

The signal measurement circuit according to embodiments of the present invention has at least the following advantages:

Due to its design using plastic films, the capacitive ECG sensor can be produced cost-effectively, in particular when the same base material is used for all the layers.

Due to the appropriate choice of the base material in the various layers, the signal measurement circuit is stable with respect to changes in humidity and to the influence of fluids.

Due to the outer sides of the sensor element being formed by films, the signal measurement circuit can be cleaned and disinfected particularly thoroughly and easily.

The materials used are at least x-ray-transparent and do not obstruct an imaging procedure carried out in parallel with the ECG acquisition.

Due to appropriate selection of the layer's base materials, the signal measurement circuit is particularly flexible and in particular moldable onto a patient's anatomy and, moreover, is not sensitive to movements.

### BRIEF DESCRIPTION OF THE DRAWINGS

The present invention is described again in greater detail hereinafter with reference to the attached figures, using exemplary embodiments. In the various figures, identical components are denoted by identical reference signs. The figures are typically not to scale. In the drawings:

FIG. 1 shows a view of a differential voltage measurement system that is arranged on a patient, in a first exemplary embodiment,

FIG. 2 shows a view of a differential voltage measurement system comprising a signal measurement circuit according to embodiments of the present invention, in each case in an exemplary embodiment of the present invention, and

FIG. 3 shows a view of a signal measurement circuit in a further exemplary embodiment.

### DETAILED DESCRIPTION

In the figures, by way of example, the point of departure is an ECG measurement system 1 used as a differential voltage measurement system 1 to measure bioelectrical signals S(k), in this case ECG signals S(k). Embodiments of the present invention are not restricted thereto, however.

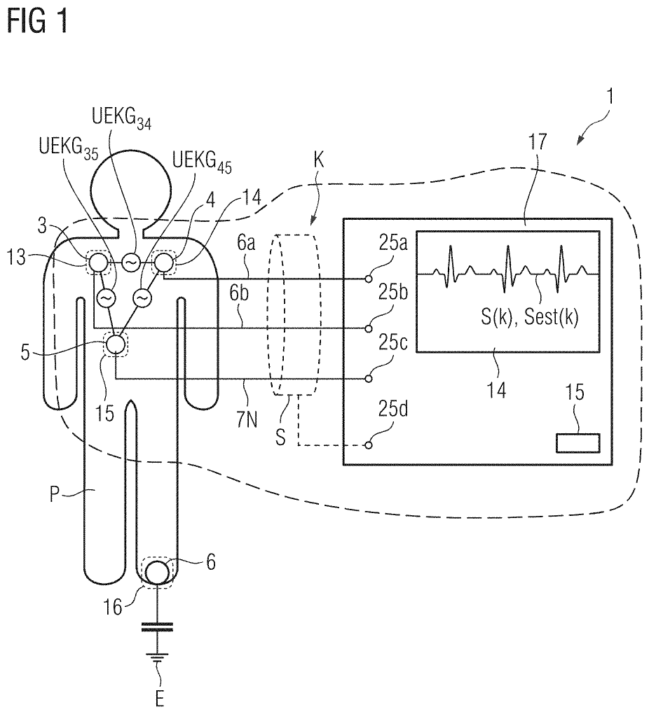

FIG. 1 shows a view of a differential voltage measurement system 1 that is arranged on a patient P, in the form of an ECG measurement system 1, in a first exemplary embodiment. The voltage measurement system 1 comprises an ECG device 17 with its electrical connections and electrodes 3, 4, 5, connected thereto via a cable K comprising in each case a sensor cable in order to measure ECG signals S(k) on the patient P.

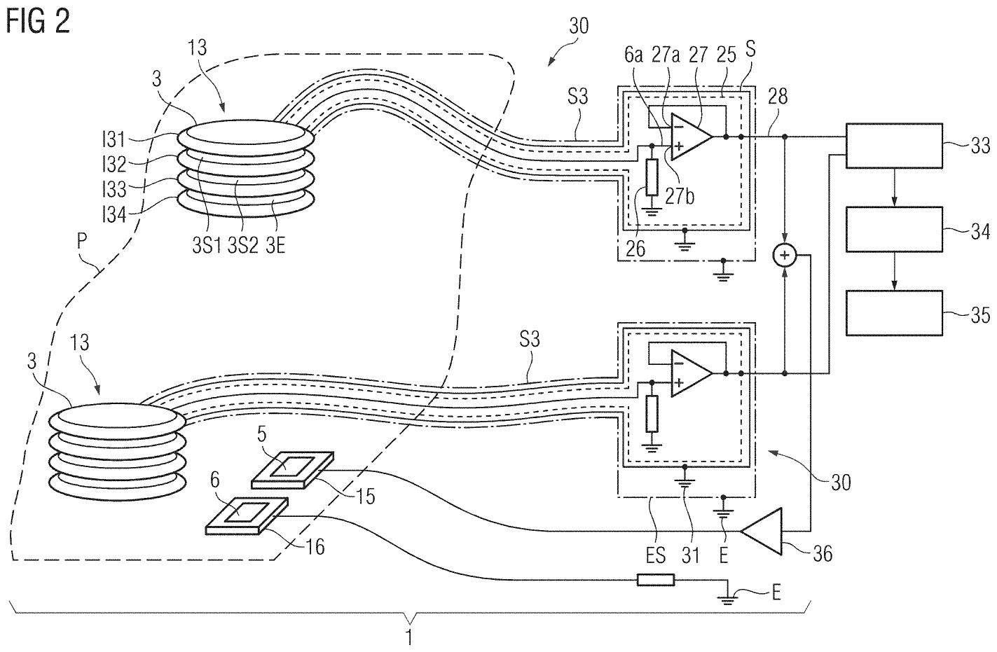

In order to measure the ECG signals S(k), according to embodiments of the present invention, at least one first sensor electrode 3 and one second sensor electrode 4, in each case formed in layers, are required, which are attached on the side of, on top of, or under the patient P. The sensor electrodes 3 and 4 are arranged in the present case on various sensor elements 13, 14. Via the signal measurement cable K, the electrodes 3, 4 are connected to the ECG device 17 by their sensor cables S3, S4 (see FIG. 2) via connectors 25a, 25b, mostly plug connectors. The first electrode 3 and the second electrode 4, including the signal measurement cable K, form part of a signal acquisition unit with which the ECG signals S(k) can be acquired.

A third electrode 5 acts as a reference electrode, in order to create a potential equalization between the patient P and the ECG device 17. This third electrode 5 is attached here via a further separate sensor element 15 close to or on the right leg of the patient P (“Right-Leg-Drive” or “RLD”).

In addition, via further connectors on the ECG device 17, which are not shown, a multiplicity of further contacts for further outlets (measurements of the potential) are attached to the patient P and used for the formation of appropriate signals. Moreover, the sensor element la can have further sensor electrodes (not shown here).

The voltage potentials UEKG34, UEKG45 and UEKG35 that are used for measuring ECG signals S(k) form between the individual electrodes 3, 4, 5.

The ECG signals S(k), which are measured directly, are displayed on a user interface 14 of the ECG device 27.

During the ECG measurement, the patient P is at least capacitively connected to the earth potential E via the ground circuit comprising a ground electrode 6 that is likewise embodied here as a separate sensor element 16.

The signal measurement cable K or the respective sensor cables S3, S4, which lead from the first sensor electrode layer 3 and the second sensor electrode layer 4 to the ECG device 17, form part of the wanted signal pathways 6a, 6b. The signal measurement cable K, which leads from the reference electrode 5 to the ECG device 17, corresponds here with part of a third wanted signal pathway 7N. The third wanted signal pathway 7N transmits interference signals, which have been coupled via the patient P and the electrodes.

The cables K have a shielding S, which is represented here in schematic form as a dotted cylinder surrounding all the wanted signal pathways 6a, 6b, 7N. However, the shielding S does not jointly surround all the cables K, unlike what is shown here, but the cables K are shielded individually. The connectors 25a, 25b, 25c preferably each have an integral pole for the shielding S. These poles are then merged onto a common shielding connector 25d. Here, the shielding S is embodied per cable K as a carbon-enriched plastic layer or plastic film surrounding the sensor cable, which layer or film extends along all the sensor cables S3, S4 up to below the layer-type sensor electrodes 3, 4.

Moreover, the ECG device 17, as shown in FIG. 1, can have an external interface 15, in order to provide, for example, a connector for a printer, a memory facility and/or even a network. The ECG device 17 also has signal measurement circuits 30 (see, for example, FIG. 2) assigned to the respective connectors 25a, 25b according to exemplary embodiments of the present invention.

FIG. 2 shows a view of a differential voltage measurement system 1 in a further exemplary embodiment of the present invention comprising two signal measurement circuits 30 in an exemplary embodiment of the present invention. The two signal measurement circuits 30 have an identical design, which is why, for reasons of clarity, corresponding components of the signal measurement circuits 30 have sometimes only been denoted by reference signs once.

The arrangement of a sensor electrode 3 is illustrated here in the form of a fundamentally capacitive ECG measurement circuit. Since the sensor electrode layer is embodied to be conductive, an ohmic coupling can also ensue in parallel with the capacitive coupling. The patient P and the sensor electrodes 3 are located in the spatial vicinity of each other. To be precise, the patient is located above the sensor electrodes 3, each of which is a component of a sensor element 13.

A sensor electrode 3 is embodied as a film-based, electrically conductive flat electrode layer 3 and has in the present embodiment a circular basic shape with an area of 25 cm². The sensor electrode layer 3 is orientated onto the patient P.

A sensor element 13, via which contact is made with the patient P, further comprises a first active shielding layer 3S1 and also a second passive shielding layer 3S2, each embodied to be conductive, which layers are arranged on the side of the sensor electrode layer 3 that faces away from the patient P. The sensor element 13 further comprises a ground electrode layer 3E that is also electrically conductive, by which the signal measurement circuit 30 according to embodiments of the present invention can be switched to the earth potential. The electrically conductive electrode and shielding layers 3, 3S1, 3S2 and 3E mentioned are separated from one another or from the surrounding environment by electrically insulating layers I31, I32, I33 and I34. To this extent, the fourth insulating layer I34 forms the outer side of the sensor element 13, which is arranged on the side of the sensor electrode layer 3 that faces away from the patient P. The insulating layers I31, I32, I33 and I34 all likewise have a circular basic shape with a diameter that is 5 mm greater than the electrode or shielding layers 3, 3S1, 3S2 and 3E, in order to safely insulate the other layers and prevent a short circuit even in the event of deformation, in particular bending of the sensor element.

The sensor element 13, with an individual-layer thickness of 50 μm in the present case, has a total height of 0.8 mm, which is advantageously thin, since it causes little disruption for the patient and is easily moldable. The individual layers are embodied in the present case from the same base material, that is, polyurethane, with carbon particles being added to the conductive electrode or shielding layers 3, 3S1, 3S2 and 3E in order to achieve a desired electrical conductivity.

In this embodiment, due to a corresponding carbon filler content, the sensor electrode layer 3 is embodied such that it has a surface resistance in the region of 100 kΩ. The first and second insulating layers I31, I32 are embodied here such that they have a volume resistance of 5 GΩ. However, the third insulating layer I33 is embodied such that it has a volume resistance of 20 GΩ.

The sensor element 13 is coupled with a measurement amplifier circuit via the sensor cable S3. The various layers 8, in the present case incorporated into the sensor element 13, merge into the sensor cable S3 at a cable input on the sensor element side and are further guided in the sensor cable S3 (with for the sake of clarity, only the conductive layers 3, 3S1, 3S2 and 3E being shown in the sensor cable S3 and further in the right-hand part of FIG. 2, and not the insulating layers that also continue along there). The sensor electrode layer 3 is provided as the core conductor element in the center of the sensor cables S3, which cables run around the core conductor through the other layers, shown here embodied as tubes in an arrangement consistent with the sensor element 13. The sensor cable S3 therefore essentially displays the layer structure of the sensor element 13.

The structure of a signal measurement circuit 30 is explained in greater detail hereinafter. Under normal conditions the patient P does not wear electrically conductive clothing. The sensor electrodes 3 can therefore couple with the patient P capacitively.

The measurement amplifier circuit, comprising an operational amplifier 27, to which the sensor cable S3 runs, is surrounded here by an active protective shield 25 forming the active shielding layer 3S1 on the sensor element 13 and by a passive protective shield S, forming the passive shielding layer 3S2 on the sensor element 13. The operational amplifier 27 is embodied as a “trailer”. This means that the negative input 27a of the operational amplifier 27 is coupled with the output 28 of the operational amplifier 27. In this way, a high virtual input impedance is achieved on the operational amplifier 27 at the positive input 27b. This advantageously achieves the result that, due to the voltage adjustment between the output 28 and the positive input 27b, hardly any current flows between the sensor electrode layer 3 and the active shielding layer 3S1 or the active protective shield 25. Furthermore, the positive input 27b of the operational amplifier 27 is kept on an electrical bias voltage with the aid of a resistor 26 switched against the measurement device ground (also known as the “measurement ground”). Hence the positive, high impedance input 27b is set at a desired measurement potential. In this way, in particular during a predominantly capacitive coupling, DC components can be suppressed.

The measured signal of the sensor electrode layer 3 is coupled onto this high impedance input 27b.

The active protective shield 25 and passive protective shield S or the active shielding layer 3S1 and passive shielding layer 3S2 completely surround the measurement amplifier circuit or completely demarcate the sensor electrode layer 3 in the direction of the surrounding environment in order to achieve effective shielding.

The passive protective shield S is likewise connected to the ground of the device 31.

Over the entire signal measurement circuit 30, both protective shields 25, S are surrounded by a ground layer 3E, which is coupled to the earth potential E. The ground layer 3E or the ground shield ES, which embodies the ground layer 3E on the sensor element 13, advantageously achieves a further improvement in the conduction of interference signals across the earth contact, an improvement in the shielding of electromagnetic external interference fields, and an improvement in the conduction of electrostatic charges.

A further surface electrode embodied as a ground electrode 6 is also provided in the separate sensor element 16 shown here, for at least capacitive and/or ohmic coupling of the patient P to the earth potential E.

A further flat electrode embodied as a reference electrode 5 or the respective measurement circuit 36 is used in the further sensor element 15 for conduction of the potential, for example as a “driven neutral electrode” (DNE).

The reference electrode 5 and ground electrode 6 are shown in the left-hand part of FIG. 2 in schematic form only. Actual size ratios and the shape of the electrode may differ in practice from this diagram.

The differential voltage measurement system 1 can optionally be a switch apparatus in the form of a switch matrix 33. Where there are a multiplicity of sensor electrodes, it serves the purpose of selecting which of the sensor electrodes are used for further signal processing, for example as a function of a patient's anatomy.

The differential voltage measurement system 1 can also be a signal processing apparatus in the form of a signal processing box 34. This is embodied to carry out pre-processing of the measured signals acquired in order to remove interfering components. The signal processing apparatus 34 can be embodied to carry out standard processing with frequency-based filters, such as band pass or band stop filters, but also an enhanced noise suppression as in German patent application DE 102019203627A, for example.

Furthermore, the differential voltage measurement system 1 can comprise a triggering apparatus 35. This is embodied to detect a heartbeat of a patient P or the heartbeat rhythm and generate control signals therefrom comprising triggering or starting time data for a medical imaging facility. Based on the control signals from the triggering apparatus 35, the imaging facility calculates the times for image data acquisition.

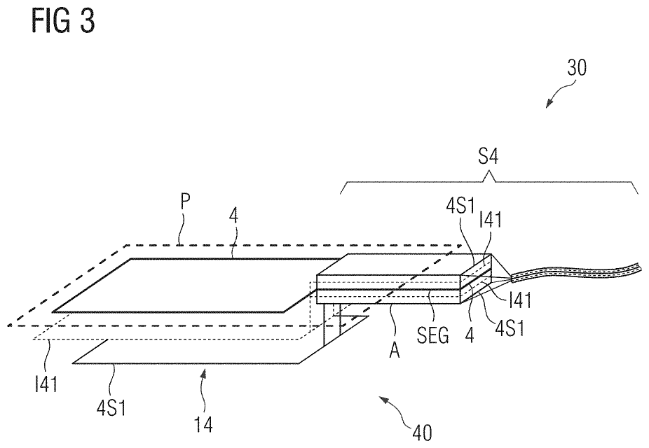

FIG. 3 shows a detailed view of a signal measurement circuit 40 according to embodiments of the present invention in a further exemplary embodiment. Here in particular, the layer-type structure of a sensor element 14 according to embodiments of the present invention is shown once again.

The signal measurement circuit 14 shown here in a layer structure for a differential voltage measurement system 1 for measuring bioelectrical signals from a patient P comprises a sensor electrode layer 4 connected via a sensor cable S4 to a measurement amplifier circuit (not shown). In the region around the sensor element 14, this sensor electrode layer is the layer turned toward the patient P, into which measured signals S(k) couple capacitively.

The sensor element 14 further comprises an active shielding layer 4S1, which runs along the side of the sensor electrode layer 4 that faces away from the patient P. The active shielding layer 4S1 serves to shield the sensor electrode layer 4 from electromagnetic interference fields from the surrounding environment. Both the sensor electrode layer 4 and active shielding layer 4S1 are electrically conductive and therefore embodied as film-type flat electrodes.

In this embodiment, the sensor element comprises at least one insulating layer I41 that is not electrically conductive, which runs between the sensor electrode layer and the active shielding layer and separates both conductive layers 4, 4S1 from each another. On the side of the active shielding layer 4S1 that faces away from the patient P, a further non-conductive insulating layer (not shown) can be provided, which forms an outer side of the sensor element 14 and delimits this in an outward direction.

In the present case, the sensor electrode layer 4 and the active shielding layer 4S1 have a rectangular cross section with a size of 30 cm² with sides measuring 6 cm times 5 cm. The rectangular shape of the conductive layers 4, 4S1 also specifies the basic shape of the sensor element 14.

The at least one insulating layer I41 and, where present, the outer insulating layer, also completely protrude here over the layers 4, 4S1 that are to be insulated and therefore have a larger area, with the sides of the insulating layer measuring 6.4 cm and 5.4 cm in order to avoid short circuits between the conductive layers 4, 4S1 in the event of movement or deformation of the signal measurement circuit 40.

The present at least 3 layers 4, I41, 4S1 of the sensor element 14 each have a layer thickness of 50 μm. Overall therefore, a thickness of the sensor element ranging from 0.15 mm to max. 0.5 mm (where there is a further outer insulating layer) is achieved, which is advantageously thin, effects a high flexibility of the signal measurement circuit 40 and does not reduce patient comfort.

The sensor electrode layer 4 and the active shielding layer 4S1 are formed here from a carbon particle-enriched plastic film, in this case PVC, in order to achieve the desired conductive properties. Here the filler content is set such that the sensor electrode layer 4 has a surface resistance of 50 kΩ.

The insulating layers, in particular insulating layer I41, likewise consist in the present case of a plastic film, but without the admixture of carbon 11. The insulating layer I41 is embodied such that it has a volume resistance of 5 GΩ.

When molding the sensor element 14 or during transport or storage of the signal measurement circuit 40, in order to avoid damage to said arrangement, the sensor electrode layer 4, the active shielding layer 4S1 and the insulating layers are welded together. This form of connection is effective over the entire surface of the layers and is hence particularly stable and easy to implement with different plastics.

Using plastics as base materials for the layer structure of the sensor element 14 is particularly advantageous since plastics have a very good mechanical durability with a high flexibility.

In the present embodiment of the signal measurement circuit 40, the sensor electrode layer 4 comprises a segment SEG that is surrounded on both sides by the insulating layer I41 and the active shielding layer S41 and in this way forms a section A of the sensor cable S4. The section A is therefore embodied in the style of a flat conductor, which has a length of 30 cm with a width of 3 cm. Where the layer thickness is 50 μm, as stated in the aforementioned, the height of section A is then 250 μm to 400 μm (or higher), which is still very thin. Since at least the sensor electrode layer 4 protrudes over the segment SEG directly into section A, the coupling between the sensor element 14 and the sensor cable S4 is preferably stable and not susceptible to interference. Since the patient's body also often protrudes into the region of the sensor cable S4 when the sensor element 14 is molded, patient comfort and the guarantee of interference-free signal transmission can be ensured via section A. After section A, the sensor cable S4 merges into a conventional core conductor element, as described with reference to FIG. 2.

Finally, it is once again pointed out that the detailed installations described in the aforementioned are merely exemplary embodiments that can be modified in a great variety of ways by a person skilled in the art without departing from the scope of the present invention. Therefore, the differential voltage measurement system can not only be an ECG device, but also other medical devices with which bioelectrical signals are acquired, such as for example EEGs, EMGs etc. Furthermore, the use of the indefinite articles “a” or “an” does not preclude the relevant features from being present in plurality.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuity such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Although the present invention has been shown and described with respect to certain example embodiments, equivalents and modifications will occur to others skilled in the art upon the reading and understanding of the specification. The present invention includes all such equivalents and modifications and is limited only by the scope of the appended claims.

Where it is not explicitly stated, yet logical and in the sense of embodiments of the present invention, individual exemplary embodiments, individual partial aspects or features thereof can be combined or exchanged without going beyond the scope of the present invention. Advantages of the present invention described with reference to one exemplary embodiment also apply, where transferable, to other exemplary embodiments without any explicit mention.

What is claimed is:

1. A signal measurement circuit in a layer structure for a differential voltage measurement system to measure bioelectrical signals from a patient, the signal measurement circuit comprising:
a sensor electrode layer connected via a sensor cable to a measurement amplifier circuit;
an active shielding layer, which runs along a side of the sensor electrode layer that faces away from the patient; and
a first insulating layer that runs between the sensor electrode layer and the active shielding layer; wherein
the sensor electrode layer and the active shielding layer are electrically conductive.

2. The signal measurement circuit as claimed in claim 1, further comprising:
a further electrically conductive shielding layer arranged on a side of the active shielding layer that faces away from the sensor electrode layer, the further electrically conductive shielding layer being separated from the active shielding layer by a second insulating layer.

3. The signal measurement circuit as claimed in claim 2, further comprising:
an electrically conductive ground electrode layer arranged on a side of the further electrically conductive shielding layer that faces away from the sensor electrode layer, the electrically conductive ground electrode layer being separated from the further electrically conductive shielding layer by a third insulating layer and being separated from a surrounding environment by a fourth insulating layer.

4. The signal measurement circuit as claimed in claim 3, wherein at least one of the sensor electrode layer, the active shielding layer, the further electrically conductive shielding layer or the electrically conductive ground electrode layer has an area ranging from 9 cm² to 64 cm².

5. The signal measurement circuit as claimed in claim 3, wherein at least one of the first insulating layer, the second insulating layer, the third insulating layer or the fourth insulating layer completely protrudes over at least one layer to be insulated.

6. The signal measurement circuit as claimed in claim 3, wherein at least one of the sensor electrode layer, the active shielding layer, the further electrically conductive shielding layer, the electrically conductive ground electrode layer, the first insulating layer, the second insulating layer, the third insulating layer or the fourth insulating layer has a layer thickness ranging from 50 μm to 500 μm.

7. The signal measurement circuit as claimed in claim 3, wherein at least one of the sensor electrode layer, the active shielding layer, the further electrically conductive shielding layer, the electrically conductive ground electrode layer or at least one of the first insulating layer, the second insulating layer, the third insulating layer or the fourth insulating layer is fused or welded with at least one adjacent layer.

8. The signal measurement circuit as claimed in claim 3, wherein at least one of the sensor electrode layer, the active shielding layer, the further electrically conductive shielding layer or the electrically conductive ground electrode layer is formed from a carbon particle-enriched plastic.

9. The signal measurement circuit as claimed in claim 1, wherein the sensor electrode layer has a surface resistance ranging from 10 kΩ to 100 kΩ.

10. The signal measurement circuit as claimed in claim 3, wherein at least one of the first insulating layer, the second insulating layer, the third insulating layer or the fourth insulating layer is formed of a plastic.

11. The signal measurement circuit as claimed in claim 2, wherein the first insulating layer and the second insulating layer have a volume resistance ranging from 50 MΩ to 50 GΩ.

12. The signal measurement circuit as claimed in claim 3, wherein the third insulating layer has a volume resistance ranging from 1 GΩ to 100 GΩ.

13. The signal measurement circuit as claimed in claim 1, wherein the sensor electrode layer has a segment that is surrounded on both sides by the first insulating layer and the active shielding layer to form a section of the sensor cable.

14. The signal measurement circuit as claimed in claim 13, wherein the section of the sensor cable has a length ranging from 20 cm to 200 cm and a width ranging from 2 cm to 6 cm.

15. A differential voltage measurement system to measure bioelectrical measured signals from a patient, the differential voltage measurement system comprising:
at least two signal measurement circuits each corresponding to a signal pathway of the differential voltage measurement system, at least one of the at least two signal measurement circuits including
a sensor electrode layer connected via a sensor cable to a measurement amplifier circuit,
an active shielding layer, which runs along a side of the sensor electrode layer that faces away from the patient, and
a first insulating layer that runs between the sensor electrode layer and the active shielding layer; wherein
the sensor electrode layer and the active shielding layer are electrically conductive.

16. The signal measurement circuit as claimed in claim 4, wherein at least one of the first insulating layer, the second insulating layer, the third insulating layer or the fourth insulating layer completely protrudes over at least one layer to be insulated.

17. The signal measurement circuit as claimed in claim 5, wherein at least one of the sensor electrode layer, the active shielding layer, the further electrically conductive shielding layer, the electrically conductive ground electrode layer, the first insulating layer, the second insulating layer, the third insulating layer or the fourth insulating layer has a layer thickness ranging from 50 μm to 500 μm.

18. The signal measurement circuit as claimed in claim 5, wherein at least one of the sensor electrode layer, the active shielding layer, the further electrically conductive shielding layer, the electrically conductive ground electrode layer or at least one of the first insulating layer, the second insulating layer, the third insulating layer or the fourth insulating layer is fused or welded with at least one adjacent layer.

19. The signal measurement circuit as claimed in claim 2, wherein the sensor electrode layer has a segment that is surrounded on both sides by the first insulating layer and the active shielding layer to form a section of the sensor cable.

20. The signal measurement circuit as claimed in claim 3, wherein the sensor electrode layer has a segment that is surrounded on both sides by the first insulating layer and the active shielding layer to form a section of the sensor cable.
